(19) United States
(12) Patent Application Publication
(10) Pub. No.: US 2008/0208181 A1
(43) Pub. Date: Aug. 28, 2008

(54) Thermally Insulated Needles For Dermatological Applications

(75) Inventors: Didier Toubia, Raanana (IL); Alexander Levin, Binyamina (IL); Miron Kaganovich, Haifa (IL)

Correspondence Address: HESLIN ROTHENBERG FARLEY & MESITI PC, 5 COLUMBIA CIRCLE, ALBANY, NY, 12203, US

(73) Assignee: Arbel Medical Ltd., Yokneam Moshava (IL)

(21) Appl. No.: 12/017,035

(22) Filed: Jan. 20, 2008

Related U.S. Application Data
(60) Provisional application No. 60/885,681, filed on Jan. 19, 2007.

(51) Int. Cl. A61B 18/02

(52) U.S. Cl. 606/20

(57) ABSTRACT

For active thermal insulation of a cryoneedle or hyperthermia needle, preferably, the device comprises a needle for transmitting heat or cold, surrounded by a sheath for providing an opposing temperature to protect surrounding skin.

### FIELD OF THE INVENTION

The present invention is of cryoprobes or hyperthermia probes for dermatological applications, and in particular to such probes which feature cryosurgical or hyperthermia needles at their distal tips.

### BACKGROUND OF THE INVENTION

Various references describe cryoprobes of small diameters (cryoneedles), while others describe cryoprobes which feature active heating of their sheaths or shafts.

Rabin (U.S. Pat. No. 6,786,902) describes an apparatus for cryosurgery. The apparatus comprises a cryoneedle having a diameter of less than 3.2 mm. This apparatus also features a thermal insulation shell disposed about a portion of the cryoneedle for reduction of heat transfer from surrounding tissues or preventing surrounding tissues from freezing during application of the cryoneedle with the shell. The cryoneedle and shell are configured for insertion into a body of a patient. However, the shell is passive and does not relate to direct heating.

Luo (U.S. Pat. No. 6,672,095) discloses a therapeutic freezing device, which includes a barrel and a superconducting needle. The barrel defines a receiving space adapted to receive a coolant medium. The superconducting needle is mounted on the barrel and is adapted to contact the coolant medium so that the low-temperature of the coolant medium is transferred to the superconducting layer. The superconducting needle includes a superconductive material.

Har-Shai (U.S. Pat. No. 6,503,246) describes intralesional method for treating a hypertrophic scar or keloid using a cryoprobe. The method comprises: inserting the cryoprobe into the hypertrophic scar or keloid so that the cryoprobe is positioned within the hypertrophic scar or keloid; and introducing a cryogen into the cryoprobe thereby freezing the hypertrophic scar or keloid. The cryoprobe has a sealed distal end comprising a cutting tip. Also disclosed is a cryoprobe comprising an elongated, uninsulated housing having a sealed distal end and a proximal end. The housing comprises therein a cryogen inlet tube. The cryoprobe further comprises a cutting tip at the distal end of the housing and a cryogen vent adjacent to the proximal end and in fluid communication with the interior of the housing.

Rabin (U.S. Pat. No. 6,039,730) discloses an apparatus for cryosurgery. The apparatus comprises a cryoneedle having a diameter less than 3.2 mm. The apparatus is also comprised of a thermal insulation shell disposed about a portion of the cryoneedle for reduction of heat transfer from surrounding tissues or freezing prevention of surrounding tissues during application of the cryoneedle with the shell. The cryoneedle and shell are configured for insertion into a body of a patient. It pertains to a method for freezing tissues. The method comprises the steps of bringing into contact a cryoneedle having a diameter of less than 3.2 mm with a patient's body. Next, there is the step of flowing the cryofluid through the cryoneedle.

In addition, cryoprobes produced by Galil Medical Company (Israel) under a name CRYOSEED with diameter of 1.47 mm may optionally be regarded as cryoneedles. These cryoprobes operate on the base of the Joule-Thomson effect.

There are also a number of U.S. patents related to active heating of a cryoprobe (or cryocatheter) shaft. For example Onik (U.S. Pat. No. 6,379,348) discloses a combined electrosurgical-cryosurgical instrument for tissue ablation. The instrument comprises a shaft having a proximal end and a distal end, the distal end being electrically and thermally conductive; a radiofrequency insulation sheath surrounding the outer surface of the shaft; a cryo-insulation sheath surrounding a surface of the shaft; a radiofrequency power supply source; a cryogen supply tube within the shaft; and a cryogen supply source connected to the cryogen supply tube. The power source provides electrical energy to the distal end of the shaft, and the cryogen supply tube provides a cryogen to the distal end of the shaft.

Maurice (U.S. Pat. No. 6,858,025) describes a cryosurgical apparatus including an elongate cryoprobe having a cooling portion and an electrically conductive first portion in the region of the cooling portion. A removable sheath having an electrically conductive second portion is received on the cryoprobe with its electrically conductive second portion spaced from the electrically conductive first portion of the cryoprobe. Electrical insulation is interposed between the first portion and the second portion. Coolant material supplied to the cryoprobe produces tissue freezing in the region of the cooling portion. Electromagnetic energy supplied to either the first portion or the second portion, while the other of such first portion or second portion is connected to ground, provides selective heating in tissue surrounding an iceball produced by the cooling portion to control the configuration of the iceball.

Maurice (US Patent Application Publication No. 2005/0038422) discloses a cryosurgical apparatus, which includes an elongate cryoprobe having an electrically conductive first portion and multiple cooling elements. A removable sheath having an electrically conductive second portion is received on the cryoprobe with its electrically conductive second portion spaced from the electrically conductive first portion of the cryoprobe. Electrical insulation is interposed between the first portion and the second portion. In operation, cooling elements in the cryoprobe cool the tissue around a portion of the cryoprobe while electromagnetic energy traveling between the first portion and the second portion heats tissue adjacent to the cooled tissue. The cooling alters the path of the electromagnetic energy by changing the electrical conductivity of the tissue in the region of the cryoprobe.

Maurice (US Patent Application Publication No. 2004/0215178) discloses a cryosurgical apparatus, which includes an elongate cryoprobe having a cooling portion and an electrically conductive first portion in the region of the cooling portion. A removable sheath having an electrically conductive second portion is received on the cryoprobe with its electrically conductive second portion spaced from the electrically conductive first portion of the cryoprobe. Electrical insulation is interposed between the first portion and the second portion. Coolant material supplied to the cryoprobe produces tissue freezing in the region of the cooling portion. Electromagnetic energy supplied to either the first portion or the second portion, while the other of such first portion or second portion is connected to ground, provides selective heating in tissue surrounding an iceball produced by the cooling portion to control the configuration of the ice-ball.

In addition, there are some U.S. patents describing hyperthermia microwave probes with needle-like antennas.

For example, Prakash (U.S. Pat. No. 7,128,739) discloses such microwave probe; however, overheating of the skin is a significant danger with such a probe.

### SUMMARY OF THE INVENTION

The present invention overcomes these disadvantages of the background art by providing, in some embodiments, a cryoprobe which features a disposable cryoneedle at its distal tip. In other embodiments, the present invention provides a hyperthermia probe with a disposable needle-like heating element on its distal end.

The technical solutions in the design of the cryoprobe and its disposable cryoneedle allow this cryoneedle to be constructed with a narrow effective diameter, which is preferably significantly less than about 1 mm. In addition, the cryoneedle includes a sheath for active thermal insulation of the upper layer of the skin; in such a way, it prevents the skin from freezing and hence prevents cryoablation from occurring in the immediate vicinity of the cryoneedle.

There is a plurality of embodiments of active thermal insulation of the cryoneedle according to the present invention. In both embodiments, the cryoneedle preferably comprises a pin constructed from a material with high thermal conductivity. The distal end of the pin is preferably pointed or sharpened in order to facilitate its penetration into the skin. The pin may optionally be fabricated from silver, gold or copper with thin layer of protective coating, or as a metal pin with a layer of a diamond film coating. A middle section of the pin is preferably surrounded with a metal sheath; the inner diameter of the metal sheath is preferably somewhat larger than the diameter of the pin, so that there is a narrow gap between the pin and the metal sheath. This gap serves as a thermal barrier, which decreases heat transfer between the pin and the metal sheath.

The sheath is secured at its distal and proximal ends with the pin with one or more thin layers of glue; in such a way, the internal gap between the pin and the metal sheath is sealed.

The distal section of the pin is preferably inserted into a blind opening in the tip of the distal section of the cryoprobe. In addition, there is preferably a radial threaded opening in this tip, which optionally allows the pin to be secured, and, therefore, the cryoneedle itself, with a small screw.

The outer lateral surface of the tip of the cryoprobe is preferably provided with a means for fastening an additional sheath; the length of this additional sheath is somewhat less than the length of the metal sheath of the cryoneedle. The additional sheath is optionally and preferably provided with a lateral manifold with a port, for supplying a warm gaseous medium into the gap between the metal sheath of the cryoneedle and the additional sheath. This warm gaseous medium serves for heating the metal sheath until a desirable temperature. In addition, if the flow rate of the warm gaseous medium is sufficiently high, it warms the skin immediately by direct contact between the skin and the warm gaseous medium. The distal edge of the additional sheath is preferably toothed to permit escape of gases.

In the second embodiment, there is a distal tubular piece, which is optionally fastened on the tip of the cryoprobe with a (preferably polymer) bushing; the internal diameter of the distal tubular piece preferably conforms to the outer diameter of the metal sheath.

A coil of thin metal wire with electrical insulation is preferably wound on the outer lateral surface of the distal tubular piece, for periodically heating the distal tubular piece and, therefore, the metal sheath by pulses of electrical current. The level of current is preferably adjustable. During intervals between the pulses, the coil may optionally be used to measure electrical resistance, for estimating the average temperature of the distal tubular piece and the metal sheath, and for adjusting the power of pulses of electrical current in order to maintain the average temperature within a desirable interval.

Optionally at least two different coils are used: one for measuring the temperature of the metal sheath and another for heating the metal sheath to a desirable temperature level.

Therefore, the distal section of the metal sheath, which penetrates into the skin during cryosurgical treatment, preferably heats the surrounding area of the skin and hence provides protection against freezing of, and damage to, surrounding tissue.

For embodiments featuring a hyperthermia needle-like probe, the section of the needle which is in immediate contact with the skin is preferably at least partially surrounded by a protecting sheath cooled by a gaseous medium to a suitable temperature.

For such embodiments, a hyperthermia probe preferably comprises an external shaft with a proximal inlet connection for electrical wires. Alternatively the shaft may comprise a proximal seat for installation of a radiation unit. A miniature bulb or a single emitter laser diode may optionally be used; in addition this radiation unit can include a cooling sub-unit. The shaft features a distal face plane tip with an outer blind hole for positioning a hyperthermia disposable needle and a blind hole for positioning a temperature measuring means. The internal surface of the distal face plane tip preferably features a coating with a high coefficient of absorption of radiation emitted by the radiation unit.

When a metal coil is optionally used as a heating source, it is preferably wound on the proximal section of the distal face plane tip and the ends of this metal coil are connected to the electrical wires.

The hyperthermia disposable needle preferably comprises a pin fabricated from a material with high thermal conductivity. The distal end of the pin is preferably sharpened or pointed in order to facilitate its penetration into the skin. The pin may optionally be fabricated from silver, gold or copper with a thin layer of protecting coating, or as a metal pin with a layer of a diamond film coating, as previously described. Again as previously described, a middle section of the pin is preferably at least partially surrounded with a metal sheath; the inner diameter of the metal sheath is preferably somewhat larger than the diameter of the pin such that there is a narrow gap between the pin and the metal sheath. This gap serves as a thermal barrier, which decreases heat transfer between the pin and the metal sheath.

The sheath is optionally secured at its distal and proximal ends with the pin by one or more thin layers of glue, such that the internal gap between the pin and the metal sheath is sealed.

The distal section of the pin is preferably inserted into a blind hole in the tip of the distal section of the cryoprobe. In addition, there is a radial blind threaded hole in this tip, which allows the pin, and, therefore, the hyperthermia disposable needle itself, to optionally be secured with a small screw.

In addition, one or more miniature thermoelectric elements may optionally be used for cooling the metal sheath of the disposable hyperthermia needle. For this embodiment, preferably a metal saddle is in good thermal contact with the metal sheath, and the thermoelectric elements are positioned on the outer surface of the metal saddle. Miniature radiators are preferably placed on the opposite sides of the thermoelectric elements and act as a heat sink.

The outer lateral surface of the tip of the hyperthermia probe optionally features means for fastening an additional sheath; the length of this additional sheath is somewhat less than the length of the metal sheath of the hyperthermia disposable needle. The additional sheath preferably features a lateral manifold with a port for receiving a cooling gaseous medium into the gap between the metal sheath of the hyperthermia disposable needle and the additional sheath. This cooling gaseous medium cools the metal sheath to a desirable temperature. In addition, if the flow rate of the cooling gaseous medium is sufficiently high, it cools the skin immediately by direct contact between the skin and the cooling gaseous medium. The distal edge of the additional sheath is preferably toothed for escape of such gases.

The distal section of the pointed pin for the both embodiments—a cryoprobe and a hyperthermia probe—may optionally be provided with a longitudinal recess, which is preferably filled with a material such as a polymer foam for example that has low thermal conductivity, such that the needle forms a lune.

According to some embodiments, there is provided a probe for thermal ablation of a tissue area of skin, comprising: a source of thermal ablation energy; an external shaft and a distal face plane tip; a needle for piercing the skin and in thermal communication with the distal face plane tip for delivering thermal ablation energy; and an external sheath surrounding at least a portion of the needle, comprising a lateral manifold with a port for delivery of a heat transfer gaseous medium.

Optionally the needle comprises a pointed pin from a material with high thermal conductivity and a sheath joined with the pointed pin, whereby a narrow gaseous gap is defined between the pointed pin and the sheath. Preferably the pointed pin is fabricated from a material with an external coating by a diamond layer.

Optionally the sheath is fabricated from a metal. Preferably the sheath is fabricated from a metal with high thermal conductivity.

Optionally the sheath is provided with a diamond coating.

Optionally, the sheath is joined to the pointed pin with one or more thin layers of glue.

Optionally and preferably the source of thermal energy comprises a cryogen in liquid or gaseous-liquid (mist) form, the probe further comprising a feeding lumen in fluid communication with the internal space of the external shaft for transmitting the cryogen.

Optionally, the external sheath includes a toothed distal edge for removal of the gaseous medium.

Optionally, the needle comprises a pin fabricated from a material with high thermal conductivity and the distal section of the pin is provided with a longitudinal lune, wherein the longitudinal lune is filled by a polymer material with low thermal conductivity. Optionally, the pin has a diameter of less than about 1 mm.

Optionally the probe further comprises an additional sheath for at least partially surrounding the pointed pin, wherein the pin and the additional sheath are joined by one or more layers of glue with formation of a gaseous sealed gap between the pin and the additional sheath.

Optionally and preferably the source of thermal energy comprises a metal coil in thermal contact with the distal face plane tip; for heating the distal face plane tip and the pin, which is in thermal communication with the distal face plane tip, and wherein the gaseous medium is a cooling medium.

According to some embodiments there is provided a probe for thermal ablation of a tissue area located in an immediate vicinity of skin, comprising: a longitudinal housing with a proximal inlet connection for electrical wires; a distal face plane tip with an outer blind hole, the distal face plane tip featuring an absorbing coating with high coefficient of absorption of radiation; a hyperthermia disposable needle positioned in the outer blind hole and comprising a pin, wherein a distal end of the pin is pointed; a metal sheath at least partially surrounding a middle section of the pin; wherein the source of thermal energy comprises a radiation source for heating, the probe further comprising a seat at the proximal end of the housing, wherein the radiation source is installed in the seat, and a concentrating lens within the housing for concentrating the energy.

Preferably the probe further comprises a plurality of metal saddles surrounding the metal sheath, a plurality of thermoelectric elements positioned on the outer surface of the metal saddles; and a plurality of miniature radiators on the opposite sides of the thermoelectric elements as heat sinks.

Optionally and preferably, the probe further comprises an additional sheath, wherein the length of the additional sheath is less than the length of the metal sheath; the additional sheath being provided with a lateral manifold with a port for providing a cooling gaseous medium into a gap between the metal sheath of the hyperthermia disposable needle and the additional sheath.

According to some embodiments there is provided a probe for thermal ablation of a tissue area located in an immediate vicinity of skin, comprising: a longitudinal housing with a proximal inlet connection for electrical wires; a distal face plane tip with an outer blind hole; a hyperthermia disposable needle positioned in the outer blind hole and comprising a pin, wherein a distal end of the pin is pointed; a metal coil connected to the electrical wires and contacting the face plane tip for heating the pin; a metal sheath at least partially surrounding a middle section of the pin; an additional sheath for at least partially surrounding the metal sheath, wherein a gap is formed between the additional sheath and the external sheath for receiving a gaseous medium for cooling.

Preferably, the metal coil serves in addition for measuring the temperature of the distal face plane tip according to the electrical resistance of the metal coil.

Preferably the metal coil is energized by pulses of electrical current.

Such embodiments provide anisotropic freezing or heating of the surrounding tissue while minimizing damage to the healthy tissue in the immediate vicinity of the distal section of the tip of the needle.

### BRIEF DESCRIPTION OF THE FIGURES

For a better understanding of the invention and to show how it may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention; the description taken with the drawings making apparent to those skilled in the art how the several forms of the invention may be embodied in practice.

FIG. 1a, FIG. 1b and FIG. 1c show axial cross-sections of an exemplary, illustrative cryoprobe, its disposable cryoneedle, and the distal section of the cryoprobe with its additional sheath providing dynamic thermal protection by a stream of a gaseous medium.

FIG. 2a, FIG. 2b and FIG. 2c show axial cross-sections of an exemplary, illustrative cryoprobe, its disposable cryoneedle, and the distal section of the cryoprobe with its additional sheath providing dynamic thermal protection by an electrically heated metal coil wound on the additional sheath.

FIG. 3a, FIG. 3b and FIG. 3c show axial cross-sections of an exemplary, illustrative hyperthermia probe with an electrical heating element, a disposable heating needle, and the distal section of the hyperthermia probe with its additional sheath providing dynamic thermal protection of the skin by a stream of a cooling gaseous medium.

FIG. 4 shows an axial cross-section of an exemplary, illustrative hyperthermia probe with a radiation unit, as a source of heating.

FIG. 5a and FIG. 5b show an axial cross-section of an exemplary, illustrative hyperthermia probe with thermoelectric elements for cooling a metal sheath of the hyperthermia needle with providing dynamic thermal protection of the skin, and a transversal cross-section of a saddle for positioning the thermoelectric elements.

FIG. 6 shows an axial cross-section of an exemplary, illustrative disposable heating needle or a disposable cryoneedle with anisotropic freezing or heating of the surrounding tissue.

### DESCRIPTION OF THE PREFERRED EMBODIMENTS

The present invention provides active thermal insulation of a cryoneedle or hyperthermia needle, for providing a cryoprobe or a hyperthermia probe. For either type of probe, a needle comprises accordingly active thermal insulation from cold or heat. The needle preferably comprises a pin constructed from a material with high thermal conductivity. The distal end of the pin is preferably pointed or sharpened in order to facilitate its penetration into the skin. The pin may optionally be fabricated from silver, gold or copper with a thin layer of protective coating, or as a metal pin with a layer of a diamond film coating.

For either heating or cooling embodiments, a middle section of the pin is optionally and preferably surrounded with a sheath which is more preferably fabricated from metal; the inner diameter of the metal sheath is preferably somewhat larger than the diameter of the pin, so that there is a narrow gap between the pin and the metal sheath. This gap serves as a thermal barrier, which decreases heat transfer between the pin and the metal sheath. More preferably, the sheath features a manifold for receiving a gaseous medium having a counteractive temperature to that of the needle, such that the gaseous medium preferably is cooler than the needle for hyperthermia applications and warmer than the needle for cryogenic applications. Thus, the sheath is preferably used for active thermal insulation of the needle apart from at the tip, thereby reducing damage to surrounding tissue.

For cryogenic applications, optionally the sheath may feature an interior coil to which electrical energy is applied, for actively heating the needle.

For hyperthermia applications, optionally one or more miniature thermoelectric elements may be used for cooling the sheath of the disposable hyperthermia needle.

The principles and operation of the present invention may be better understood with reference to the drawings and the accompanying description.

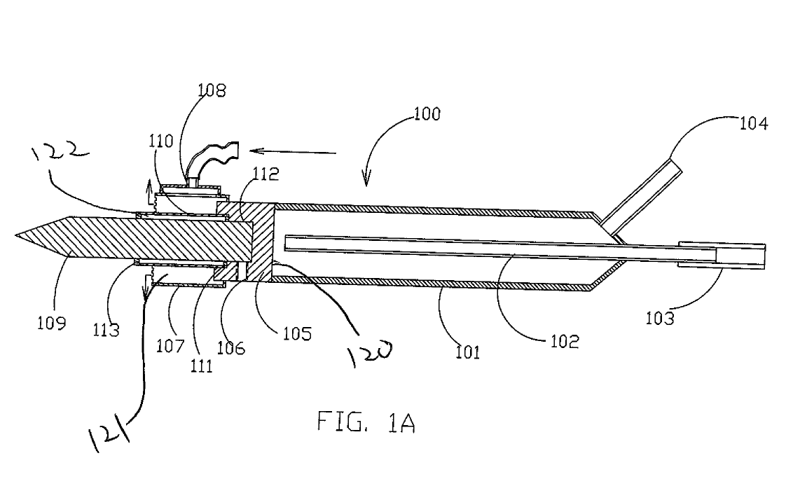

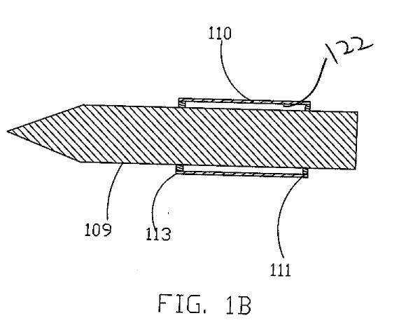

Referring now to the drawings, FIG. 1a, FIG. 1b and FIG. 1c show axial cross-sections of a cryoprobe, its disposable cryoneedle, and the distal section of the cryoprobe with its additional sheath providing dynamic thermal protection with a gaseous medium.

The cryoprobe 100 comprises: an external shaft 101; an internal feeding lumen 102, an inlet connection 103; an outlet connection 104; and a cryotip 105 with a blind hole 112 for receiving a pointed pin 109, which is preferably constructed from metal. A radial blind hole 106 with threading preferably receives a screw (not shown) for fixing the pointed pin 109. Inlet connection 103 is preferably connected to a source of cryogen (not shown), which then enters internal feeding lumen 102 for boiling at a distal end, thereby cooling cryotip 105 and hence pin 109. Cryogenic gases may then exit from outlet connection 104.

In addition, the cryoneedle preferably includes a sheath 110 for active thermal insulation of the upper layer of the skin (not shown) which is preferably constructed from metal; this sheath 110 is joined with the pointed pin 109, optionally by solid glue joints 111 and 113 with formation of a narrow gap 122 between the pointed pin 109 and the sheath 110.

There is preferably an additional sheath 107 with the proximal edge fastened on the cryotip 105. The additional sheath 107 features a lateral manifold with port 108, for supplying a warm gaseous medium into the gap 121 between sheath 110 of the cryoneedle and the additional sheath 107. This warm gaseous medium serves for heating sheath 110 to a desirable temperature. The distal edge of the additional sheath 107 is provided with a plurality of teeth 114 for exhausting the heating gas to the surroundings via the gaps between the teeth 114. Teeth 114 of the additional sheath 107 preferably contact the surrounding tissue (not shown).

In operation, pointed pin 109 is inserted into the skin to the desired depth of cryo-treatment. The previously described source of cryogen (not shown) is connected to inlet connection 103, such that cryogen enters through inlet connection 103 and hence to internal feeding lumen 102 for boiling at a face plane 120 (shown in FIGS. 1A and 1C), thereby cooling cryotip 105 and hence pin 109. Cryogenic gases may then exit from outlet connection 104. To prevent damage to surrounding tissue, a warm gaseous medium is supplied through port 108 and hence to the gap 121 between the sheath 110 of the cryoneedle and the additional sheath 107. The surrounding tissue contacts the additional sheath 107, which is warmed by the gaseous medium and which is therefore substantially or completely undamaged by the cryogenic treatment, while sheath 110 maintains a colder temperature for pin 109. Additional sheath 107 preferably features a plurality of teeth 122 to permit the gaseous medium to exit.

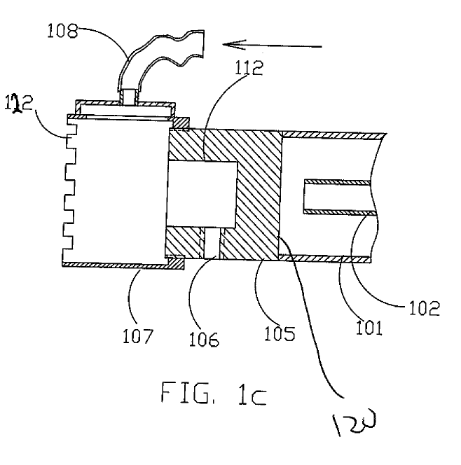

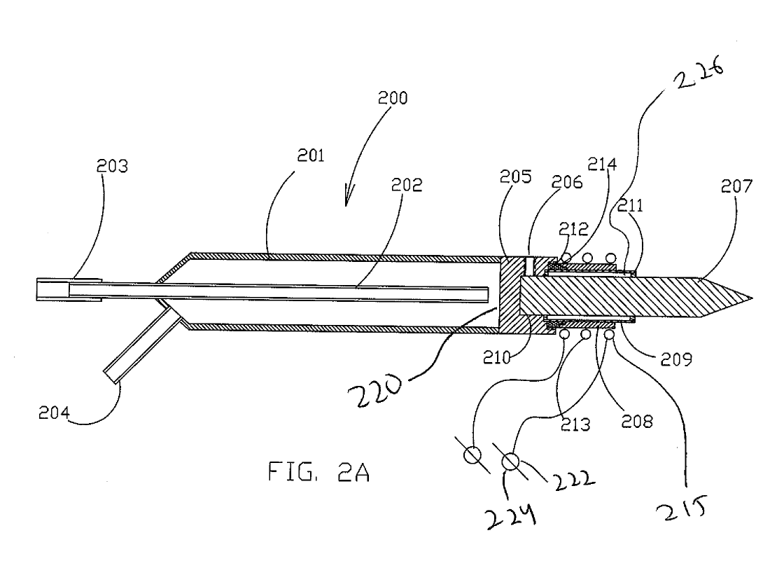

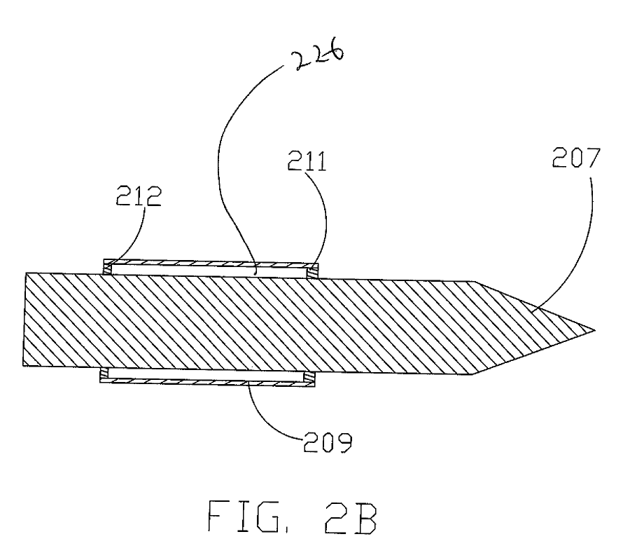

FIG. 2a, FIG. 2b and FIG. 2c show axial cross-sections of a cryoprobe, its disposable cryoneedle, and the distal section of the cryoprobe with its additional sheath providing dynamic thermal protection by an electrically heated metal coil wound on the additional sheath.

The cryoprobe 200 comprises: an external shaft 201; an internal feeding lumen 202, an inlet connection 203; and an outlet connection 204; all of which function substantially as described with regard to FIG. 1. Cryoprobe 200 also preferably features a face plane cryotip 205 with a blind hole 210 to receive a pointed pin 207, which is preferably constructed from metal. A radial hole 206 with threading preferably receives a screw (not shown) for fixing the pointed pin 207.

In addition, the cryoneedle includes a sheath 209, preferably constructed from metal, for active thermal insulation of the upper layer of the skin; this sheath 209 is joined with the pointed pin 207, optionally by solid glue joints 212 and 211, with formation of a narrow gap 226 between the pointed pin 207 and the sheath 209.

Furthermore a distal tubular piece 208 is preferably provided and separated from the face plane cryotip 205 by a (preferably polymer) bushing 214. The distal tubular piece 208 has an internal diameter which provides a forced fit for the external diameter of the sheath 209, for ensuring effective thermal contact between these elements. The outer surface of the distal tubular piece 208 preferably features a coil of thin metal wire 213 with an electrical insulation 215; this coil 213 heats the tubular piece 208, and, therefore, the external tissue (not shown). Preferably heating is performed periodically with pulses of electrical current with proper adjustable power; during periods between the pulses, electrical resistance of coil 213 is optionally and preferably measured with a measuring device 222 at a power source 224 (show schematically).

In operation, pointed pin 207 is inserted into the skin to the desired depth of cryo-treatment. The previously described source of cryogen (not shown) is connected to inlet connection 203, such that cryogen enters through inlet connection 203 and hence to internal feeding lumen 202 for boiling at a face plane 220 (shown in FIGS. 2A and 2C), thereby cooling cryotip 205 and hence pin 207. Cryogenic gases may then exit from outlet connection 204. To prevent damage to surrounding tissue, coil 213 heats the tubular piece 208. The surrounding tissue contacts the tubular piece 208, which is warmed by the gaseous medium and which is therefore substantially or completely undamaged by the cryogenic treatment, while sheath 209 maintains a colder temperature for pin 207. Temperature is optionally determined by measuring resistance of the coil 213 as described above.

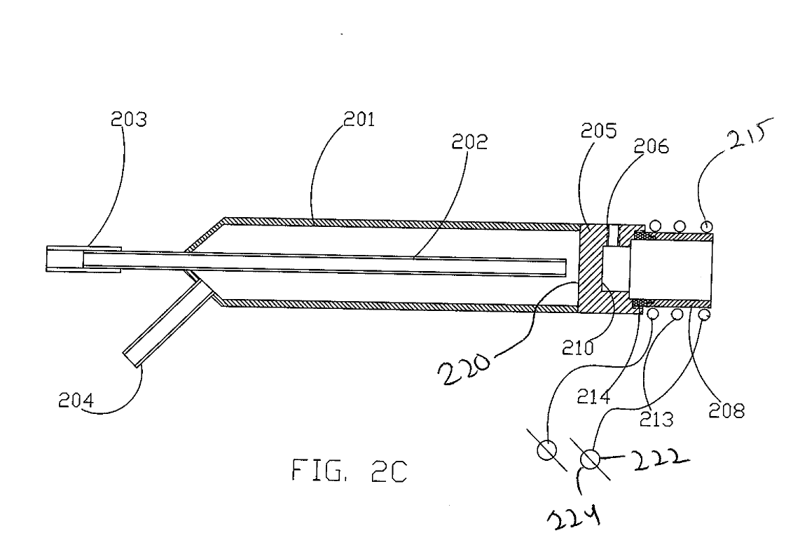

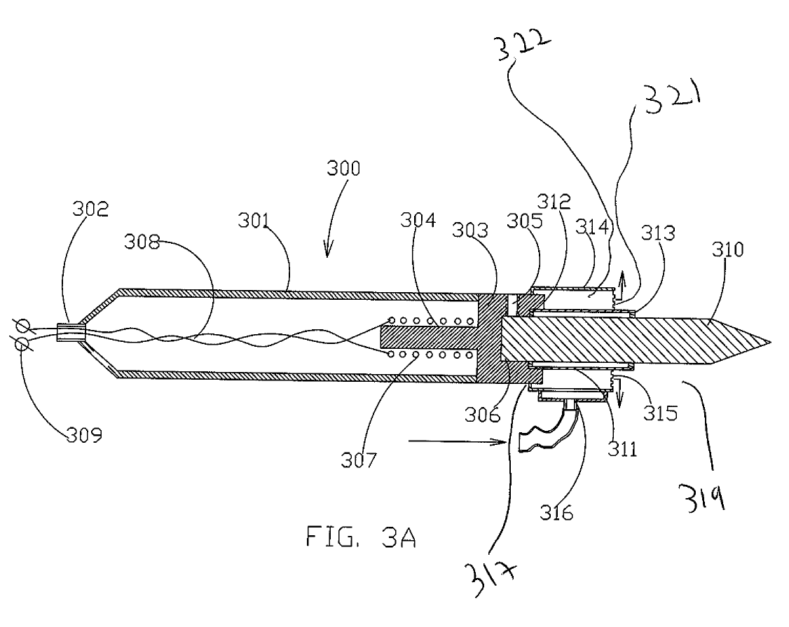

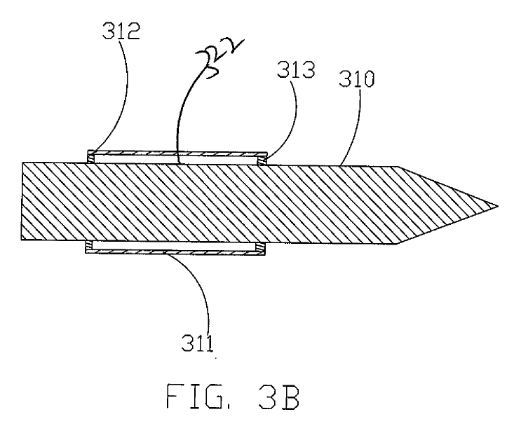

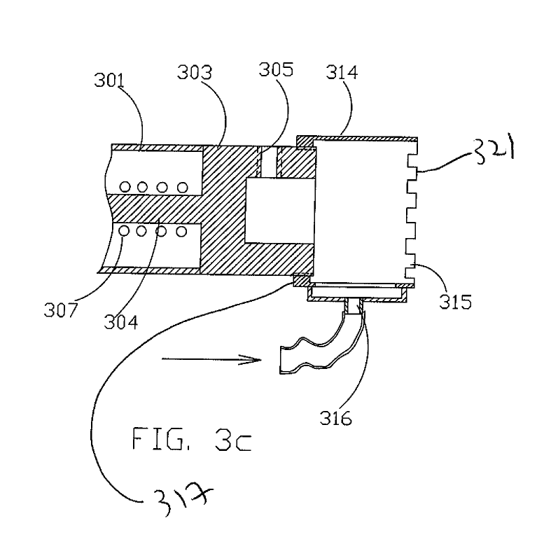

FIG. 3a, FIG. 3b and FIG. 3c show axial cross-sections of a hyperthermia probe, its disposable heating needle, and the distal section of the hyperthermia probe with its additional sheath providing dynamic thermal protection with a gaseous medium. In this embodiment, dynamic thermal protection is provided to prevent excessive heating of surrounding tissue.

A hyperthermia probe 300 preferably comprises a longitudinal housing 301 with a proximal inlet connection 302 for electrical wires 308 with connection sockets 309; and a distal face plane tip 303 with an outer blind hole 306 for positioning a hyperthermia disposable needle 319.

A proximal section 304 of the distal face plane tip 303 is provided with a metal coil 307, which is connected with the electrical wires 308 for heating this face plane tip 303 upon passing electrical current through electrical wires 308. In addition, the metal coil 307 may optionally be used to measure the temperature of the distal face plane tip 303 according to the electrical resistance of this metal coil 307.

The hyperthermia disposable needle 319 preferably comprises pin 310 which is preferably fabricated from a material with high thermal conductivity. The distal end of pin 310 is pointed in order to facilitate its penetration into the skin (not shown). A middle section of the pin is preferably at least partially surrounded with a metal sheath 311. The metal sheath 311 is preferably secured at its distal and proximal ends to pin 310, optionally with layers of glue 312 and 313.

For operation, pin 310 is inserted into the outer blind hole 306 of the distal face plane tip 303 of the hyperthermia probe 300. In addition, there is optionally a radial blind threaded hole 305 in this distal face plane tip, for securing pin 310 by a small screw (not shown).

The outer lateral surface of the distal face plane tip 303 of the hyperthermia probe is provided with a fastener 317 for fastening an additional sheath 314; the length of this additional sheath 314 is somewhat less than the length of the sheath 311 of the hyperthermia disposable needle 319. The additional sheath is provided with a lateral manifold with port 316 for providing a cooling gaseous medium into the gap 322 between the sheath 311 of the hyperthermia disposable needle 319 and the additional sheath 314. The distal edge 315 of the additional sheath 314 preferably features a plurality of teeth 321 to permit escape of gases.

In operation, pointed pin 310 is inserted into the skin to the desired depth of heat treatment. A power source (not shown) is connected to connection sockets 309, such that electrical power is supplied to wires 308, thereby heating metal coil 307 and hence pin 310. To prevent damage to surrounding tissue, a cooling gaseous medium is supplied through port 316 and hence to the gap 322 between the sheaths 311 of the hyperthermia disposable needle 319 and the additional sheath 314. The surrounding tissue contacts the additional sheath 314, which is cooled by the gaseous medium and which is therefore substantially or completely undamaged by the heat treatment, while sheath 311 maintains a warmer temperature than pin 310. The gaseous medium may exit through distal edge 315 and may therefore optionally provide additional cooling of the tissue (not shown).

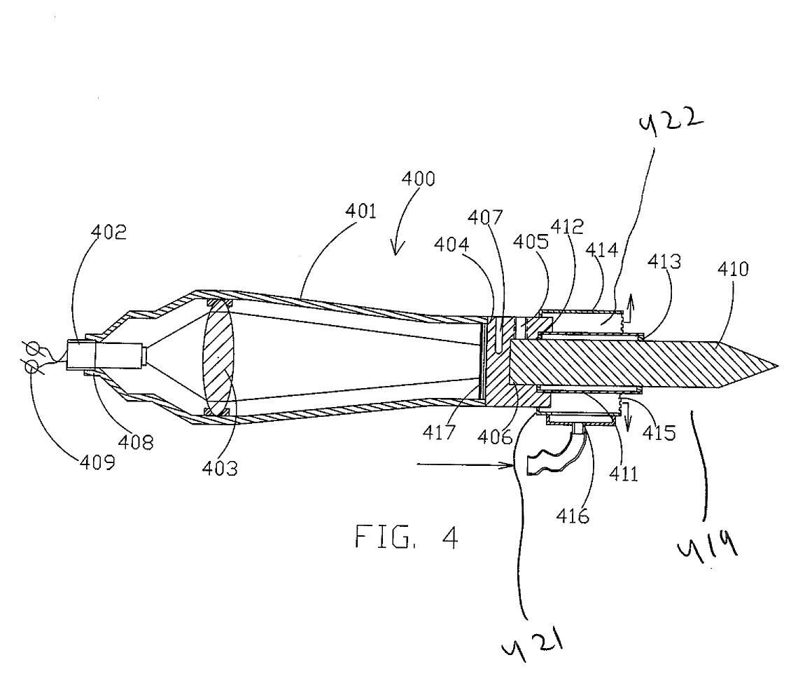

FIG. 4 shows an axial cross-section of a hyperthermia probe with a radiation unit and a disposable heating needle.

The probe 400 comprises a longitudinal housing 401 with a proximal seat 408 for a radiation source 402, a concentrating lens 403 and connection sockets 409; a distal face plane tip 404 is preferably provided with an outer blind hole 406 for positioning a hyperthermia disposable needle 419. Radiation source 402 is preferably a source of radiation energy (for which energy is received from connection sockets 409), which is then concentrated by concentrating lens 403 onto an internal surface of the distal face plane tip 404, which is preferably provided with an absorbing coating 417 with a high coefficient of absorption of radiation of the type emitted by the radiation unit 402.

The hyperthermia disposable needle 419 preferably comprises pin 410 fabricated from a material with high thermal conductivity for receiving heat through absorbing coating 417. The distal end of pin 410 is pointed in order to facilitate its penetration into the skin (not shown). A middle section of the pin is surrounded with a sheath 411, which is preferably constructed from metal. The sheath 411 is preferably secured at its distal and proximal ends to pin 410, optionally with layers of glue 412 and 413.

Pin 410 is inserted into the outer blind hole 406 of the distal face plane tip 404 of the hyperthermia probe 400. In addition, there is a radial blind threaded hole 405 in this distal face plane tip, for securing pin 410 with a small screw (not shown).

The outer lateral surface of the distal face plane tip 404 of the hyperthermia probe is preferably provided with a fastener 421 for fastening an additional sheath 414; the length of this additional sheath 414 is somewhat less than the length of the metal sheath 411 of the hyperthermia disposable needle. The additional sheath is provided with a lateral manifold with port 416 for providing a cooling gaseous medium into the gap 422 between the metal sheath 411 of the hyperthermia disposable needle and the additional sheath 414, thereby cooling the surrounding skin as a specific tissue area is heated with pin 410. The distal edge 415 of the additional sheath is toothed for permitting escape of such gases.

A radial blind hole 407 in housing 401 may optionally be used to secure a handle (not shown).

In operation, pointed pin 410 is inserted into the skin to the desired depth of heat treatment. A power source (not shown) is connected to connection sockets 409, such that power is supplied to radiation source 410, which is focused onto absorbing coating 417 by concentrating lens 403. Absorbing coating 417 is thereby heated and in turn heats pin 410. To prevent damage to surrounding tissue, a cooling gaseous medium is supplied through port 416 and hence to the gap 422 between the sheath 411 of the pin 410 and the additional sheath 414. The surrounding tissue contacts the additional sheath 414, which is cooled by the gaseous medium and which is therefore substantially or completely undamaged by the heat treatment, while sheath 411 maintains a warmer temperature than pin 410. The gaseous medium may exit through distal edge 415 and may therefore optionally provide additional cooling of the tissue (not shown).

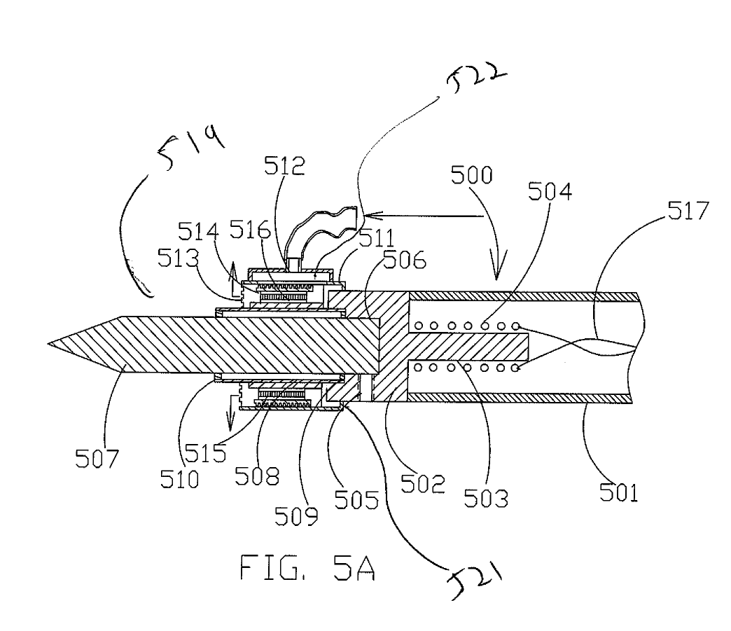

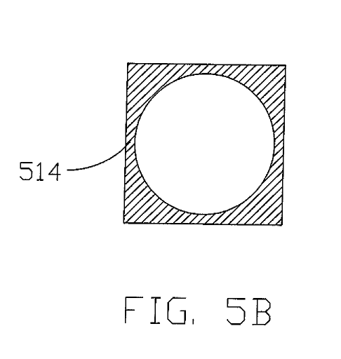

FIG. 5a and FIG. 5b show an axial cross-section of a hyperthermia probe with thermoelectric elements for cooling an additional sheath providing dynamic thermal protection of the skin, and a transversal cross-section of a saddle for positioning the thermoelectric elements.

A distal section 500 comprises a distal section of a longitudinal housing 501; and a distal face plane tip 502 with an outer blind hole 506 for positioning a hyperthermia disposable needle 519.

A proximal section 503 of the distal face plane tip 502 is provided with a metal coil 504, which is connected with the electrical wires 517 for heating this face plane tip 502. The operation is similar to that of FIG. 3.

In addition, the metal coil 504 can serve for measuring the temperature of the distal face plane tip 502 according to the electrical resistance of this metal coil.

The hyperthermia disposable needle 519 preferably comprises a pin 507 fabricated from a material with high thermal conductivity. The distal end of pin 507 is pointed in order to facilitate its penetration into the skin. A middle section of the pin is surrounded with a sheath 508, which is preferably fabricated from metal. The sheath 508 is preferably secured at its distal and proximal ends to pin 507, optionally with layers of glue 509 and 510.

Pin 507 is inserted into the outer blind hole 506 of the distal face plane tip 502 of the hyperthermia probe. In addition, there is a radial blind threaded hole 505 in this distal face plane tip, for securing pin 507 with a small screw (not shown).

The outer lateral surface of the distal face plane tip of the hyperthermia probe 500 is provided with a fastener 521 for fastening an additional sheath 511; the length of this additional sheath 511 is somewhat less than the length of the sheath 508 of the hyperthermia disposable needle 519. The additional sheath is provided with a lateral manifold with port 512, for providing a cooling gaseous medium into the gap 522 between the metal sheath 508 of the hyperthermia disposable needle and the additional sheath 511. The distal edge 513 of the additional sheath 511 is preferably toothed for permitting gas to escape.

In addition, miniature thermoelectric elements 515 are preferably applied for cooling the sheath 508 of the disposable hyperthermia needle 519. A plurality of saddles 514, which are preferably fabricated from metal and which are in good thermal contact with this sheath 508 and the thermoelectric elements 515, are preferably positioned on the outer surface of sheath 508 for conducting thermal energy away from sheath 508. Miniature radiators 516 are placed on the opposite sides of the thermoelectric elements 515 and serve as heat sinks to dissipate thermal energy from saddles 514, thereby cooling at least an external surface of sheath 508.

In operation, pointed pin 507 is inserted into the skin to the desired depth of heat treatment. A power source (not shown) supplies electrical power to wires 517, thereby heating metal coil 504 and hence pin 507. To prevent damage to surrounding tissue, saddles 514 conduct heat away from an external surface of sheath 508, with cooling provided by miniature thermoelectric elements 515 with radiators 516, thereby cooling additional sheath 511. Additional sheath 511 is also preferably cooled by gaseous medium entering through port 512. The surrounding tissue contacts the additional sheath 511, and is therefore substantially or completely undamaged by the heat treatment, while sheath 508 maintains a warmer temperature for pin 507. The gaseous medium may exit through distal edge 513 and may therefore optionally provide additional cooling of the tissue (not shown).

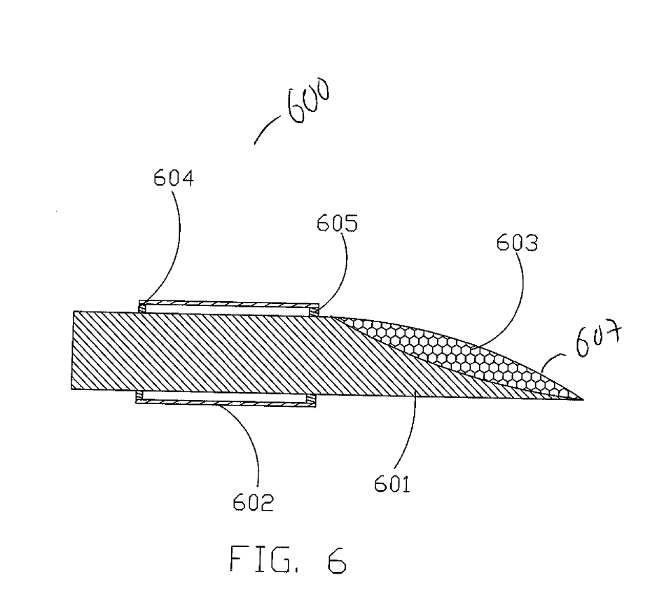

FIG. 6 demonstrates an axial cross-section of a hyperthermia disposable needle (or a disposable cryoneedle) with anisotropic heating (or freezing) of the surrounding tissue, which may optionally be used with any of the preceding embodiments. This disposable needle 600 preferably comprises a pin 601 fabricated from a material with high thermal conductivity. The distal end of pin 601 is pointed in order to facilitate its penetration into the skin (not shown). A middle section of the pin is surrounded with a sheath 602, preferably fabricated from metal.

The distal section of the pin 601 is provided with a longitudinal lune 607, which is preferably filled by a polymer material 603 with low thermal conductivity, thereby providing additional insulation for preventing damage to surrounding tissues. An external sheath 602 is preferably fastened to pin 601, optionally with glue layers 604 and 605.

While the invention has been described with respect to a limited number of embodiments, it will be appreciated that many variations, modifications and other applications of the invention may be made.

What is claimed is:

1. A probe for thermal ablation of a tissue area of skin, comprising:
a source of thermal ablation energy;
an external shaft and a distal face plane tip;
a needle for piercing the skin and in thermal communication with said distal face plane tip for delivering thermal ablation energy; and
an external sheath surrounding at least a portion of said needle, comprising a lateral manifold with a port for delivery of a heat transfer gaseous medium.

2. The probe of claim 1, wherein said needle comprises a pointed pin from a material with high thermal conductivity and a sheath joined with said pointed pin, whereby a narrow gaseous gap is defined between said pointed pin and said sheath.

3. The probe of claim 2, wherein said pointed pin is fabricated from a material with an external coating by a diamond layer.

4. The probe of claim 2, wherein said sheath is fabricated from a metal.

5. The probe of claim 4 wherein said sheath is fabricated from a metal with high thermal conductivity.

6. The probe of claim 4, wherein said sheath is provided with a diamond coating.

7. The probe of claim 2, wherein said sheath is joined to said pointed pin with one or more thin layers of glue.

8. The probe of claim 1, wherein said source of thermal energy comprises a cryogen in liquid or gaseous-liquid (mist) form, the probe further comprising a feeding lumen in fluid communication with the internal space of said external shaft for transmitting said cryogen.

9. The probe of claim 1, wherein said external sheath includes a toothed distal edge for removal of said gaseous medium.

10. The probe of claim 1, wherein said needle comprises a pin fabricated from a material with high thermal conductivity and the distal section of said pin is provided with a longitudinal lune, wherein said longitudinal lune is filled by a polymer material with low thermal conductivity.

11. The probe of claim 10, wherein said pin has a diameter of less than about 1 mm.

12. The probe of claim 2, further comprising an additional sheath for at least partially surrounding said pointed pin, wherein said pin and said additional sheath are joined by one or more layers of glue with formation of a gaseous sealed gap between said pin and said additional sheath.

13. The probe of claim 2, wherein said source of thermal energy comprises a metal coil in thermal contact with said distal face plane tip; for heating said distal face plane tip and said pin, which is in thermal communication with said distal face plane tip, and wherein said gaseous medium is a cooling medium.

14. A probe for thermal ablation of a tissue area located in an immediate vicinity of skin, comprising:
a longitudinal housing with a proximal inlet connection for electrical wires;
a distal face plane tip with an outer blind hole, said distal face plane tip featuring an absorbing coating with high coefficient of absorption of radiation;
a hyperthermia disposable needle positioned in said outer blind hole and comprising a pin, wherein a distal end of said pin is pointed;
a metal sheath at least partially surrounding a middle section of said pin;
wherein said source of thermal energy comprises a radiation source for heating, the probe further comprising a seat at the proximal end of said housing, wherein said radiation source is installed in said seat, and a concentrating lens within said housing for concentrating said energy.

15. The probe of claim 13, further comprising a plurality of metal saddles surrounding said metal sheath, a plurality of thermoelectric elements positioned on the outer surface of said metal saddles; and a plurality of miniature radiators on the opposite sides of said thermoelectric elements as heat sinks.

16. The probe of claim 15, further comprising an additional sheath, wherein the length of said additional sheath is less than the length of said metal sheath; said additional sheath being provided with a lateral manifold with a port for providing a cooling gaseous medium into a gap between said metal sheath of said hyperthermia disposable needle and said additional sheath.

17. A probe for thermal ablation of a tissue area located in an immediate vicinity of skin, comprising:
a longitudinal housing with a proximal inlet connection for electrical wires;
a distal face plane tip with an outer blind hole;
a hyperthermia disposable needle positioned in said outer blind hole and comprising a pin, wherein a distal end of said pin is pointed;
a metal coil connected to said electrical wires and contacting said face plane tip for heating said pin;
a metal sheath at least partially surrounding a middle section of said pin;
an additional sheath for at least partially surrounding said metal sheath, wherein a gap is formed between said additional sheath and said external sheath for receiving a gaseous medium for cooling.

18. The probe of claim 17, wherein said metal coil serves in addition for measuring the temperature of said distal face plane tip according to the electrical resistance of said metal coil.

19. The probe of claim 18, wherein said metal coil is energized by pulses of electrical current.
